(19) United States
(12) Patent Application Publication
(10) Pub. No.: US 2003/0013191 A1
(43) Pub. Date: Jan. 16, 2003

(54) Prostatic cell line and use thereof to obtain an established prostatic cancer in an animal

(76) Inventors: Olivier Cussenot, Paris (FR); Jean-Marie Villette, Villiers sur Marne (FR)

Correspondence Address: Lerner, David, Littenberg,, Krumholz & Mentlik, LLP, 600 South Avenue West, Westfield, NJ, 07090-1497, US

(21) Appl. No.: 10/081,348

(22) Filed: Feb. 22, 2002

Related U.S. Application Data
(63) Continuation of application No. PCT/FR00/02362, filed on Aug. 23, 2000.

(30) Foreign Application Priority Data
Aug. 23, 1999 (FR) 9910700

(51) Int. Cl. C12N 5/08

(52) U.S. Cl. 435/366

(57) ABSTRACT

The invention relates to a method for producing a non-human A mammal with a prostatic tumor. The invention also relates to a prostatic tumor and an established cell line which can form a prostatic tumor in dogs after grafting has occurred. The invention further relates to specific monoclonal antibodies of prostatic tumoral cells and to the use thereof in diagnosis or therapy.

The present invention concerns a new established cellular line of dog prostatic cancerous epithelial cells, in an animal to which have been grafted cells from this cellular line generating an established prostatic tumour and to identification processes of therapeutic substances for the prevention and the treatment of the prostate cancer.

The prostate cancer in man is a rapidly growing pathology, and today constitutes at least 85,000 new cases per year in Europe. The current treatments of locally advanced and metastatic cancers are constituted by surgical or medical castration combined or not with antiandogen prescription; nevertheless, it is a palliative treatment because it becomes ineffective within an average time of 12 to 36 months, constituting the release phase of the hormonal treatment of the disease. In this phase of the release of the hormonal treatment, the other recognised therapeutics, chemotherapy, metabolic irradiation, etc., improve the quality of life of the patients but do not alter the fatal development of the disease.

Different techniques have been developed to follow the possible therapeutic effect of a treatment, such as described above, of prostatic tumours. This follow up consists essentially of measuring the specific antigens level of the epithelial cells of the prostrate in the blood. When the level of these antigens increases, it can be the reflection of an abnormal increase of the number of prostatic epithelial cells, a sign of a tumorous progression. Amongst these antigens, the PSA (for prostate specific antigen) is the most used antigen tracer. It is a member of the family of kalikreins.

Numerous publications relate to diagnostic or prognosis tracers of the presence of a prostatic tumour. Nevertheless these always present the doctor with an uncertainty as to the distinctive character of these tracers for a malignant tumour or a benign tumour, which makes the establishment of therapeutic protocol difficult.

More recently, another specific antigen of the membrane of prostatic epithelial cells, the PSMA (for antigen membrane specific prostate) has received great attention from the scientific and medical community in so far as it is a question of a new antigen independent and specific of prostatic epithelial cells. This antigen is expressed in normal and malignant cells. It is a hydrolase folate, an essential enzyme of the metabolism of the prostatic cell. This enzyme has been cloned and sequenced (1) and its use, or the use of specific monoclonal antibodies of this antigen, enable the diagnostic and the therapeutic follow up of the treatments to be refined, in particular by scanning.

If the existence of specific tracers of prostatic epithelial cells allow a follow up of the treatments (surgical, radiotherapy, hormonal and chemotherapy treatments), there does not exist today an animal model miming in a reliable manner the prostatic tumour in man and enabling potentially active substances to be screened by measurement not only of the level of specific tracers in the blood but also by histopathological examination and by measurement of the volume of the tumour.

Even if there are established prostatic cells useable in screening processes of potentially active substances, such as those described particularly in the patent application WO 98/05797, the skilled worker knows by experiment that the screening tests on the cellular lines in vitro or in cutaneous xenograft on murine models, if they are essential at first, are largely insufficient subsequently for a therapeutic efficiency of a candidate molecule to be measured in the active element status of new drugs within the framework of a pre-clinical technique.

The canine prostate is considered as being a good model for human prostate studies in so far as the canine and human glands are morphologically similar and have a predisposition to the malign or benign transformation. It is one of the non human prostates which develops spontaneous carcinoma and the one which has an identical development to that of man. The prostate cancer in dogs is clinically aggressive, with frequent metastasis on the regional lymphatic ganglions, the bone and the lungs. In addition, high prostatic intra-epithelial neoplasia nidi (PIN), although being an intermediate stage in the progression of the normal epithelium to the carcinoma, have been found in the majority of canine cancerous prostates (J. W. Aquilia et al (1998) The Prostate 36:189-193). High grade PINs in the dog are morphologically and histologically similar to the human PIN with a rupture of the basal cell layer, a raising of the pullulative index and of the micro vascular density.

The prostatic carcinoma is most of the time diagnosed in old country dogs and if the age of the dog is converted to the physiological age of man, the average age of a prostatic diagnosis in the dog is very close, i.e. 70 years and 67 years respectively for the dog and for the man.

These considerations being made, it should prove be necessary to constitute an animal model on which the results in terms of doses and of efficiency should be able to be transposed to the man without too much difficulty.

The present invention concerns a production process of a non human mammal animal A carrier of a prostatic tumour caused after grafting in the prostate of the aforesaid animal of 10⁸ to 10⁹ cells of an established cellular line, obtained after putting into culture and mechanical separation of cells of a spontaneous prostatic tumour existing in an animal B of the same species or of a different species.

In order that in line established cells in a prostate of an animal take in the best conditions, it is preferable that the animal from which the prostatic tumorous cells are removed and the receiving animal are of the same species. Taking account of what has been said above, between the etiological similarity of prostatic tumours in the dog and in the man, the choice of the dog as the animal to establish a prostatic tumour model enabling treatment products and methods to be tested appears as particularly appropriate.

In the invention's process, the grafting in the animal's prostate of previously established in line tumorous cells must be permanent, in other words not to risk undergoing a graft reject. To that end, the animal is treated by an immunosuppressive drug, as for example cyclosporin, simultaneously with or before the grafting of the aforesaid line cells. When the cyclosporin is used, it is administered to the animal in a dose of between 1 and 10 mg per kilo and per day. The immunosuppressive drug preferably begins at least two days before the grafting of the aforesaid cells, and preferably at least five days.

The present invention also concerns an established cellular line obtained after separation and putting into culture of cells of a spontaneous prostatic tumour existing in an animal, the cells of the aforesaid line being able to be grafted in the prostate of an animal of the same species or of a different species, and bearing essential characteristics of the human prostatic tumorous epithelial cells.

For the reasons explained above, it is preferable that the donor animal, i.e. from which the prostatic cells are removed and established in line, and the receiving animal are of the same species; preferably this species is the dog so as to constitute an animal model useable in pre-clinical tests. By useable, is understood the reliability of the potential transposition into man.

The cellular line is established by removing an established prostatic tumour in a dog, mechanical separation of the tumour and putting into culture in flasks containing an appropriate nutritive environment. After propagation of the culture in this environment, the cells are treated with trypsin/EDTA. After a certain number of passages, the cells are then progressively adapted to the culture in the same nutritive environment.

In the invention, every particular attention has been concerned with the characteristics of the established line in culture, as well as the prostatic tumour obtained after grafting of the cells of the line in the dog's normal prostate.

The essential characteristics of an established line in accordance with the invention and obtained after separation of a dog's prostatic tumour then put in culture are on the one hand, that the karyotype is not less than 60 chromosomes, and, on the other hand, that the doubling time, between 20 and 35 hours is not modified by the presence of dihydrotesterone whatever is the concentration of this latter. In addition, the line in accordance with the invention does not form agar colonies.

The cellular lines in accordance with the invention and the prostatic tumours obtained after grafting of 10⁷ to 10⁹ cells of the aforesaid line have common cytological and histochemical characteristics characterising the prostatic epithelial cells' cancer.

a) The first important characteristic is the recognition of tumorous cells by a human anti-PSMA monoclonal antibody. The PSMA or specific membranous antigen of the prostrate is a new tracer expressed by the epithelial cells of the normal, hyperplastic or cancerous prostrate. The PSMA is a transmembranous glycoprotein of which almost 95% is situated outside the cell. This protein has been discovered thanks to a monoclonal antibody, called 7E11-C5.3, produced by Horoszewicz et al (Anticancer Research, 1987, 7: 927-936) against a membranous preparation coming from the cancerous prostatic line LNCap. L'AND complementary to the PSMA has been cloned by Israeli et al (Cancer Research. 1993, 53: 227-230) which has enabled its primary structure to be deduced in amino acids. The PSMA is constituted from 750 amino acids of which the 19 N-terminals are intracellular, the following 24 transmembraneous and the remaining 707 extra cellular. The potential interest of this new prostatic tracer is that it appears over expressed in the prostatic cancer and more particularly in the not very dissociated and metastatic carcinomas as well as in the prostatic cancerous cells after an androgeno-suppressive therapeutic (Wright et al., Urilogical Oncology, 1995, 1: 18-28). The existence of a human anti-PSMA monoclonal antibody recognising the human PSMA and also recognising the canine PSMA enables the identification and the follow up of the development of the cancerous cells which constitutes a quality insurance of the animal model in accordance with the invention. Indeed, the more the animal model resembles the specific biological elements of the prostate common with man, the more the extrapolation of the obtained results will be reliable.

The present invention also concerns a human anti-PSMA monoclonal antibody, called PSM-P12 and registered with the CNCM on the Aug. 6, 1999 under the number I-2280. This antibody has been produced against a peptide corresponding to the amino acids 44 to 62 (cys-lys-ser-asn-glu-ala-thr-pro-lys-his-asn-met-lys-ala-phe-leu) and localised in the N-terminal part of the extra cellular structure of the PSMA. It has been selected for its capacity to trace the normal human prostatic epithelial cells by immuno-histochemistry. This antibody specifically recognises the canine prostatic cancerous cells of the animal model. It also recognises the cells of the human prostatic line LNCaP described by Horoszewicz et coll. (Cancer Research, 1983, 43: 1809-1818). On the other hand, this antibody does not recognise the cells of human lines not expressing the PSMA like line DU-145 described by Stone et coll. (International Journal of Cancer, 1978, 21: 274-281). On the other hand, a cellular line such as the line PC-3 described by Kaighn et coll. (Investigations in Urology, 1979, 17: 16-23) expresses a PSM membranous antigen having a partial homology with the PSMA of the LNCaP line, is partially recognised by the anti-PSMA antibodies produced against the same PSMA N-terminal part of the extra cellular part of the antigen.

All monoclonal antibodies having the same PSMA eptitoptic recognition characteristics must be considered as a functional equivalent of that.

b) the established cellular lines in accordance with the invention and the prostatic tumours stemming from the grafting of the line in a canine prostate have also as common characteristic to be recognised by antibodies of the cyto keratin 19 and of the vimentin. As an example, the anticytokeratin antibody 19 is produced by a hybridoma A53-B/A2 and sold by the Sigma Company (Saint Louis, Mo.). The anti-vimentin antibody used can be a mouse monoclonal antibody such as that referenced NCL-VIM-V9 marketed by Novocastra Laboratories Ltd, Newcastle upon Tyne, UK.

In a preferred way the lines and the tumour obtained in the animal have also as characteristic of containing antigens recognised by the antibodies directed against the human antigen Ki67 and/or against the human PSA antigen. The antigen Ki67 is a cellular proliferation tracer which is preferentially found on the transformed cells, As an example the anti PSA antibody can be the polyclonal antibody A0562 marketed by Dako (Glostrup, Denmark).

c) The cells and the prostatic tumour obtained by grafting of cells are not recognised by the monoclonal antibodies directed against the cytokeratine 18 (Dako) nor against androgenic receptors of human prostatic epithelial cells.

An established cellular line in accordance with the invention is the line DPC-1 registered in the CNCM the Aug. 6, 1999 under the number I-2279. This line has a doubling time of 27 hours, which is not modified by the presence of di-hydrotesterone with different concentrations. It does not form agar colonies. Its caryotype is from 67 to 70 chromosomes in place of the normal 78 chromosomes in the canine cellular lines. Its tumour regenerability is 100% in the bare mouse in 3 to 5 weeks. The set of immuno-imaging and histo-chemical characteristics of the line is described in the example below. The present invention also concerns a non human mammal animal bearing a prostatic tumour likely to be obtained after grafting on the prostate of the said animal of 10⁸ to 10⁹ cells of an established cellular line after putting into culture of a prostatic tumour. Preferably coming from the same animal species, mechanically separated then trypsinized after several passages in a nutritive environment. The preferred species in question is the dog. The prostatic tumour caused in this animal in accordance with the invention has the same characteristics as that of the cellular line in accordance with the invention. These characteristics are common to a dog prostatic tumour and to a human prostatic tumour. The animal in accordance with the invention, and preferably the dog, therefore constitutes an excellent laboratory model reproducing the characteristics of the human prostatic tumour and therefore in fact a tool of choice in the pre-clinical experimentations of substances liable to treat the prostatic cancer in man and in the dog.

It is also one of the objects of the present invention to supply a method for identifying a substance susceptible to treating a prostate tumour, the aforesaid method including administering the effective doses of the aforesaid substance to an animal and the detection and the measurement by comparison with a substance not suspected of having a therapeutic effect of an effect on a reduction of the aforesaid tumour.

The animal in question in this method is a carrier animal of a prostatic tumour, itself developed by grafting of cells of a previously established line, the aforesaid line coming from the setting in culture of a previously developed prostatic tumour in an animal preferably of the same species as the animal to which cells are grafted. In a preferred way, the animal in question is the dog taking into account the similarities of the phenotypic and histological characters of the prostatic tumours in the dog and the man.

By effective does, it is understood, as in any screening method, the administration of a dose likely to have a preventive or curative effect on the development of a cancer of the prostate.

By substance, is understood any substance of potential therapeutic interest which is for example an organic substance based on different basic chemical backbones or of biological macromolecules having an effect of repression or inhibition of the expression of specific genes of the cancer of the prostate; that can be lastly a vector or a viral particle carrier of a suitable sequence of interest for the genic therapy of this type of cancer.

By “specific gene of the prostate”, is understood here a gene whose expression is limited to the prostate cells and more particularly to its epithelial cells, and whose expression is generally undetectable in normal cells derived from other tissues than those of the prostate. In a general way, and as the knowledge of the etiology of the development of the cancer of the prostate makes it possible to make the assumption that such or such candidate could allow to make regress a tumour or transform a tumorous cell into normal cell. The method in accordance with the invention which makes use of an animal model representative of that which could occur in the man would be able therefore to be used in pre-clinical tests.

The effect of the effect of a substance on the tumour can be measured by any known means available to the skilled worker. It can be for example immuno-imaging or histological examinations of a biopsy of the tumour. The immuno-imaging has the advantage of enabling the use of a specific monoclonal antibodies range or other antigen, itself characterising the state of the prostatic epithelial cell.

In particular, the detection and the measurement of the possible effect of a substance can be carried out by the use of a human anti PSMA monoclonal antibody, in particular the antibody PSM-P12 registered in the CNCM the Aug. 6, 1999 under the number I-2280.

The identification method of a substance of therapeutic interest in accordance with the invention, i.e. likely to treat a tumour of the prostate is also applicable to an active element such as described above (chemical substance, vector or virus for the genic therapy, etc.) coupled to a ligand of a specific receptor of tumorous cells of the prostate. This ligand can have the advantage of targeting the potential therapeutic substance of interest to its target, by sparing the cells which do not carry the receptor of the aforesaid ligand. By coupling is understood any type of coupling which is covalent or electrostatic. The covalent connection, if need be hydrolysable, will be preferred.

An interesting candidate as ligand is a specific monoclonal antibody of a surface antigen of the prostatic cell. Taking account of the properties and specificities of anti-PSMA antibodies described above, the antibody PSM-P12 is suitable to the looked for specificity. It has in addition been observed that this antibody had the capacity of internalising in the cell a substance which is coupled to it.

The invention also concerns the coupling product between a substance likely to destroy or cure the constituent transformed epithelial cells of the cancer of the prostate or of metastases of it, and a specific ligand of the aforesaid cells.

It concerns more particularly the coupling product between the antibody PSM-P12 and a substance of therapeutic interest for the cancer of the prostate.

The invention also concerns a process of obtaining a drug for the prophylaxis or the treatment of tumours of the prostate or of metastatic transformed cells of it, characterised in that as an essential constituent of the aforesaid drug the coupling product between the antibody PSM-P12 and a substance of therapeutic interest is implemented. As a substance of therapeutic interest can be included both chemical substances, radio active isotopes as well as substances obtained by genetic recombination techniques. Within the framework of the prostate cancer therapy, the hormones of type GNRH or their analogues are suitable. The skilled worker can also envisage the coupling of the complex constituted from a genic therapy product and its vector. It can then be a question of a plasmide carrier of the substance which is wished to be expressed in the diseased prostatic cells, or of defective viruses used in this type of therapy. For a review of the different means used in this respect, one can refer to “Gene delivery systems” OECD documents, 1996, 2 rue André-Pascal, 75775 Paris, Cedex 16, France. In fact, the antibody PSM-P12 proves to be an excellent drugs targeting tool.

The skilled worker will obviously understand that any type of monoclonal antibody, or more widely of ligand of a specific antigen of the prostate epithelial cells, and having as a characteristic of internalising molecules which are attached to it after connection with the specific receptor of the aforesaid ligand at the surface of the cell, is a functional equivalent of the PSM-P12 monoclonal antibody in this type of application.

In the identification method of a substance of therapeutic interest, or in the manufacturing process of a drug, the incorporation with the coupling product of a second antibody of anti-idiotype type can be envisaged. In the present case, by anti-idiotype antibody is understood any antibody, preferably monoclonal, which has a specific affinity for the conformational site constituted by the connection between the first antibody, in particular PSM-P12 and its ligand. The incorporation of such an antibody has a double advantage: the first is that its presence can prevent the internalisation in the cell of the coupling product between the first antibody and the substances of therapeutic interest, thus preventing the destruction or the metabolisation of the aforesaid substance when it acts as mediator through membrane effect. The second advantage applies more particularly when one makes use of an identification method of a therapeutic substance, in so far as any non specific fixing of the first antibody can then be eliminated, since the first antibody is only recognised by the second antibody when it is fixed by affinity with the membranous antigen of the prostatic cells.

In the identification process of substances of therapeutic interest, the antibodies can be traced by any means known to the skilled person at the time of its implementation. It can be radioactive isotopes, such as technitium 99 coupled by the Bolton-Hunter method (ref), fluorochromes, enzymes, gluteraldehyde, periodate, FITC or TRITC methods, all these well known techniques being described in Harlow E et al, “Antibodies: A Laboratory Manual”, Cold Spring Harbor, N.Y., 346-355 (1988); Voller et al, Bull, World Health Organ, 53, 55 (1976); Avrameas et al, Scand. J. Immunol., 8, Suppl. 7, 7 (1978); Wilson et al (Immunfluorescence and Related Staining Techniques”, Elsevier/North Holland Biomedical Press, Amsterdam, 215 (1978); Hijmans et al, Clin. Exp. Immunol., 4, 457 (1969) and Goding et al, J. Immunol. Meth., 13, 215 (1976).

In other words, the specificity of antibodies recognising all or part of amino acids 44 to 62 of the PSMA, make these latter a tool of choice in their use:

Either directly to identify the carrying epithelial cells of this antigen,

Or coupled with a substance of therapeutic interest, on the one hand to select this latter, on the other hand by treating the cells, by taking advantage of its internalisation capacities and its specificity.

In the first case, the use of an anti-idiotype antibody such as defined above can enable the specificity of the coupling product to be increased and prevent its internalisation in the cells.

More generally, the present invention supplies a reliable animal model of the prostate cancer, a screening process of drugs likely to treat prostate tumours. It also supplies an established cellular line stemming from a dog prostatic tumour, and able to be grafted in the prostate of a healthy animal leading to the formation of a stable tumour. The present invention also supplies a specific monoclonal antibody of the PSMA antigen and which has the characteristics, on the one hand, of being specific to prostatic epithelial cells and, on the other hand, having the capacity to internalise a substance to which it will be attached, thus enabling to target in a specific way a substance of therapeutic interest in the prostate cells. For the first time, the inventors therefore supply a global system enabling to be carried out at the same time a pre-clinical experimentation to validate the dose and the efficiency of an active principle of a future drug, and to select new drugs which could be used in the therapeutic arsenal to fight against the prostate cancer.

Experimental Part

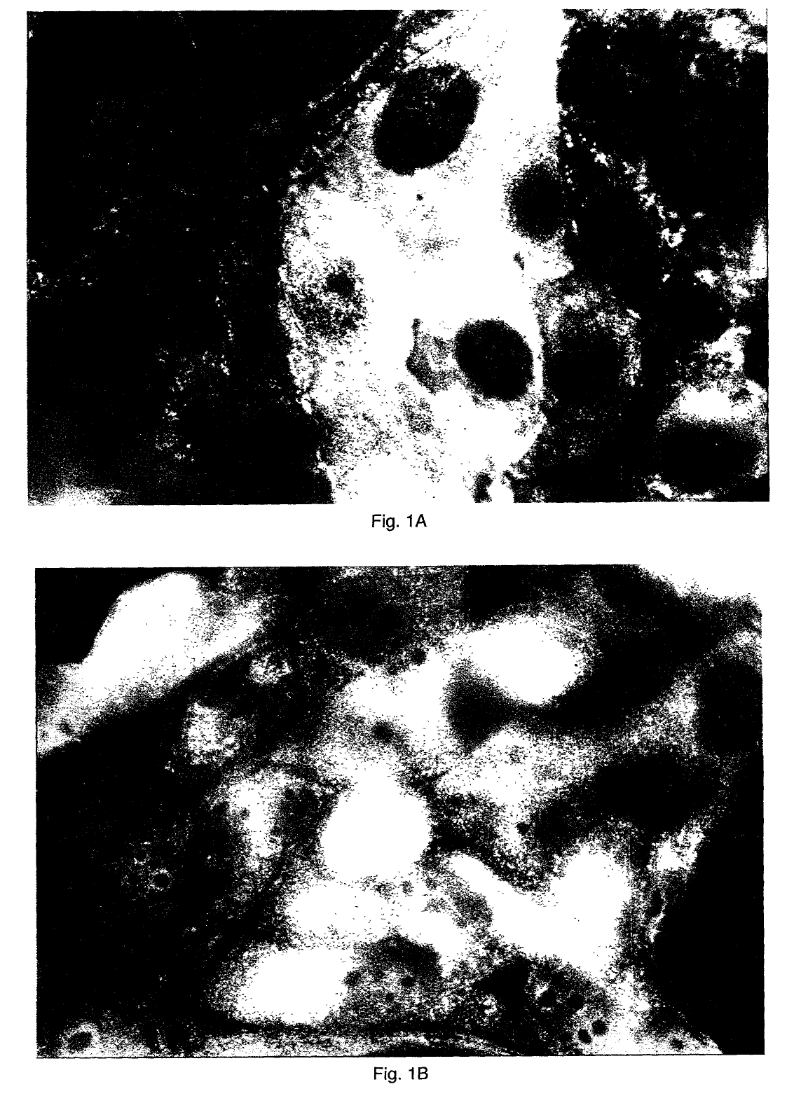

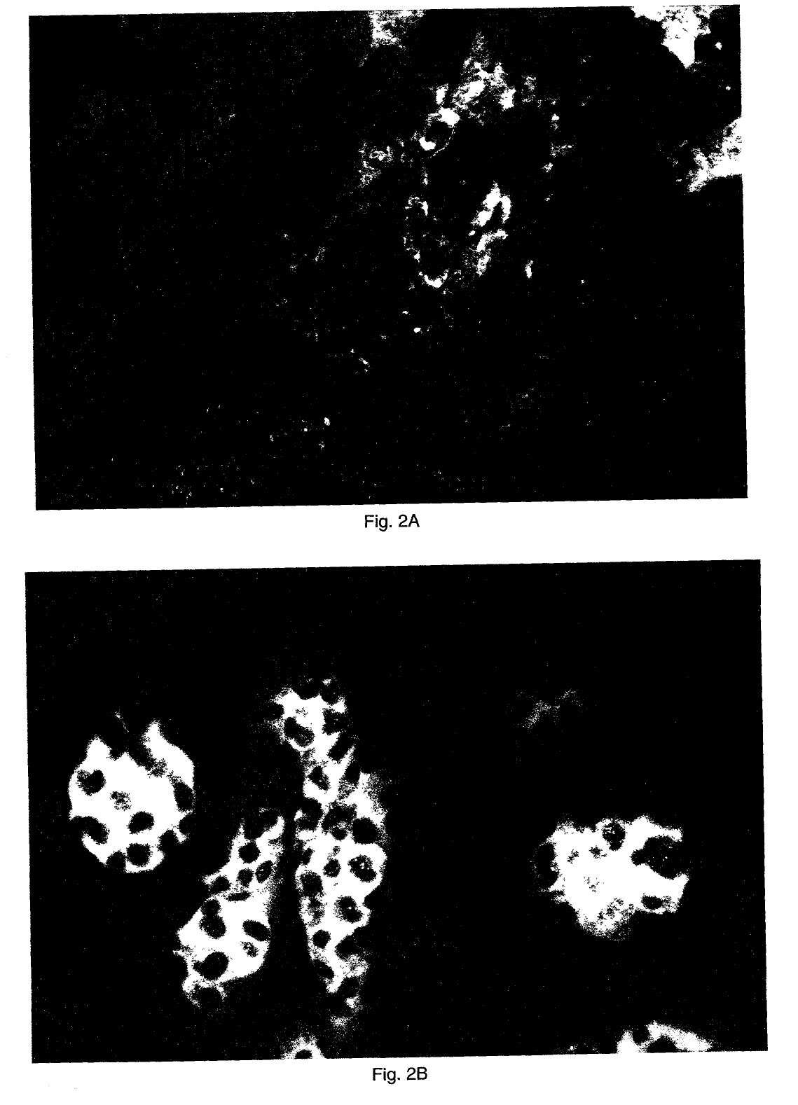

The achievement methods described in the experimental part below clarified by FIGS. 1 to 5, illustrate, without restricting it, the process and the products in accordance with the invention.

The figures have the following significance:

FIG. 1: FIG. 1 shows a positive immunofluorescence tracing of the DPC-1 line:

with a human anticytokeratine 19 mouse monoclonal antibody with 10 g/ml (FIG. 1a), released by an anti-mouse rabbit polyclonal antibody traced by the fluorescence;

with a human anti-PSA rabbit polyclonal antibody with 10 g/ml (FIG. 1b), released by an anti-rabbit mouse polyclonal antibody traced by the fluorescence.

FIG. 2: comparison of the reactivity of the LNCaP human line and the DPC-1 line regarding their reactivity vis-à-vis the human anti-PSMA P12 antibody. FIG. 2a shows the positive immunofluorescence tracing of the DPC-1 line with the human anti-PSMA mouse monoclonal antibody with 10 g/ml, released by an anti-mouse rabbit polyclonal antibody traced by the fluorescence. FIG. 2b is a tracing in identical experimental conditions of the LNCaP human line with the human anti-PSMA P12 mouse monoclonal antibody.

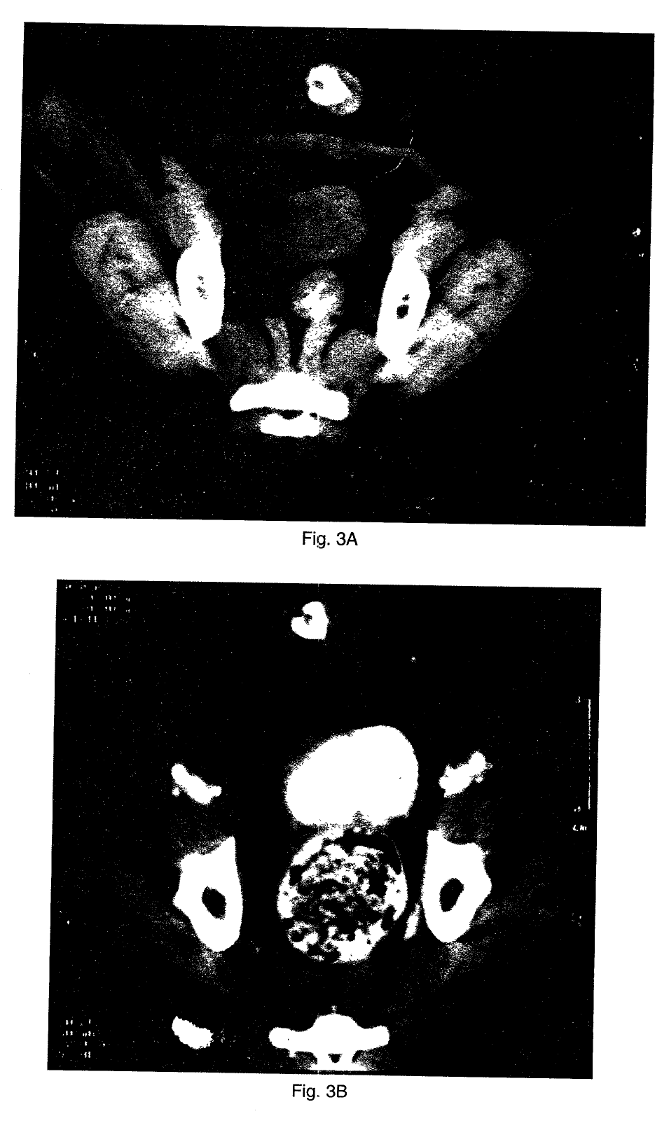

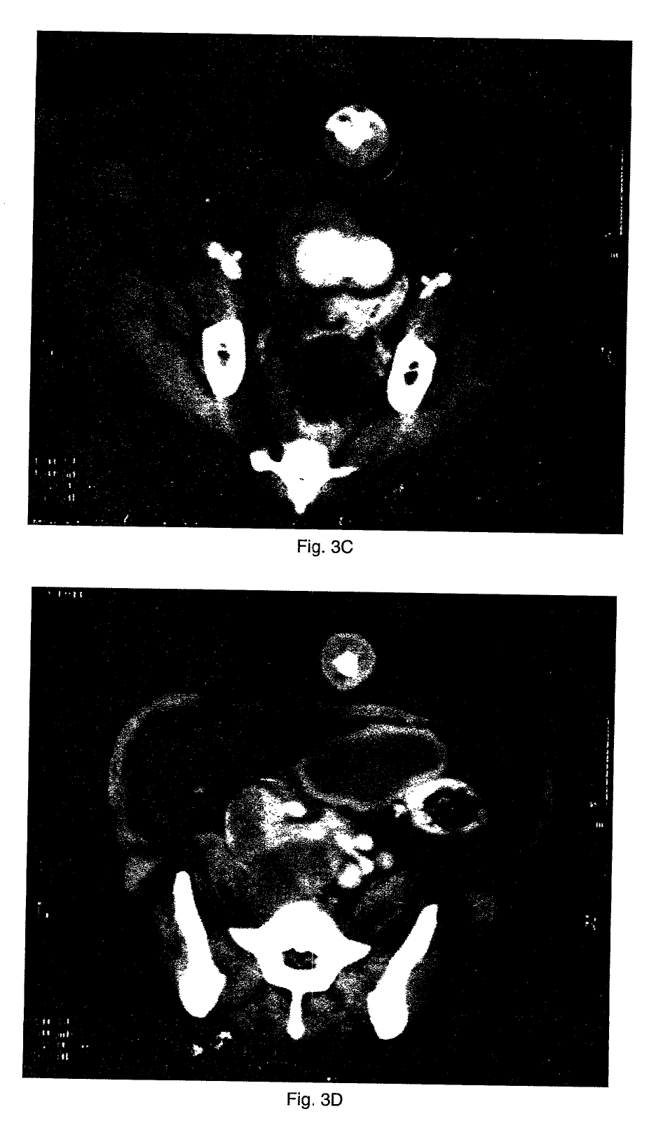

FIG. 3 shows the CT scanner images of the tumour after injection of the DPC-1 line cell into the canine prostate. FIGS. 3a and 3b show respectively the CT scanner image in transverse section of the orthopaedic DPC-1 canine model respectively at J-0 and J-14. The left lobule shows at the injection site a triangular hypodense zone with external base with three air bubbles (3a) and a retractile aspect with a hypodense tissular crown (FIG. 3b). FIG. 3c is a CT scanner image (scanner with contrast products injection) in transverse section of the DPC-1 orthotopic model at ten weeks. A significant hypodense tissular crown surrounds three quarters of the prostate. FIG. 3d is also a CT scanner image taken in the same conditions, in transverse section at the iliac level of the orthotopic DPC-1 canine model at ten weeks. A heterogeneous ganglionic voluminous mass adhering to the vertebral bone plane is visible at the left iliac level.

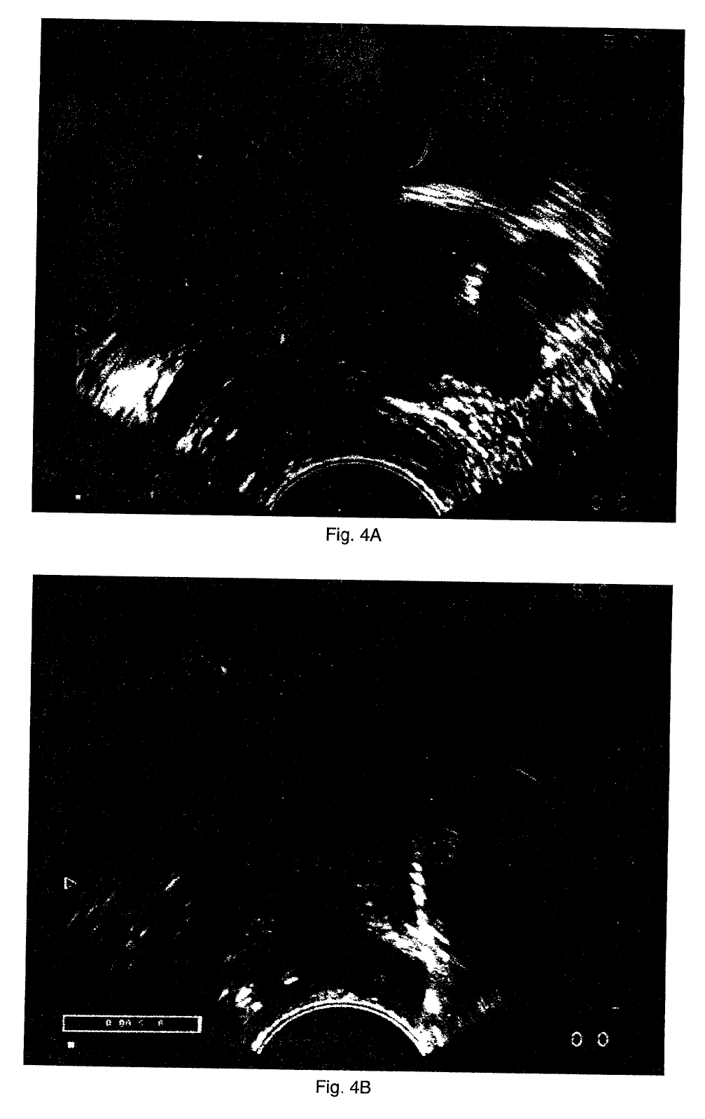

FIG. 4: this figure shows two ultrasound images of the endorectal prostate of the DPC-1 orthotopic canine model at two months. In FIG. 4a, it can be observed that the left lobule shows a peripheral hypodense zone as well as a central hypodense image. In FIG. 4b, the hypodense line (white) indicates the biopsy needle penetrating at the level of the suspect peripheral hypodense zone.

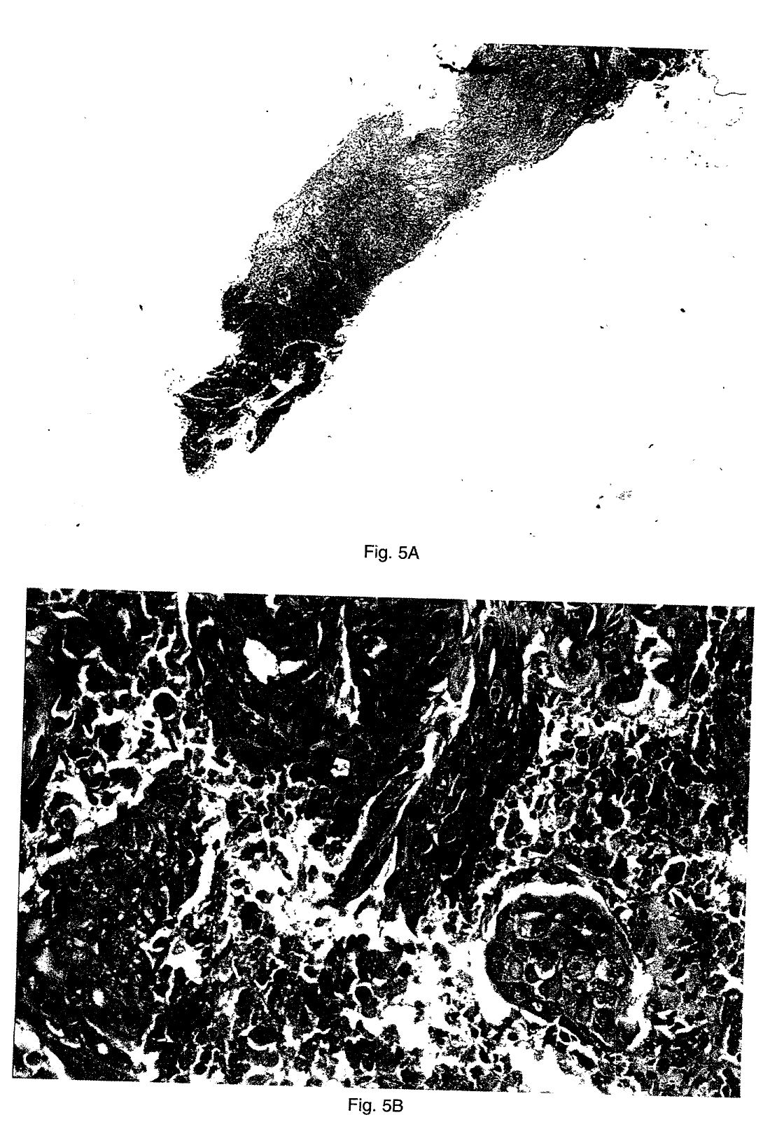

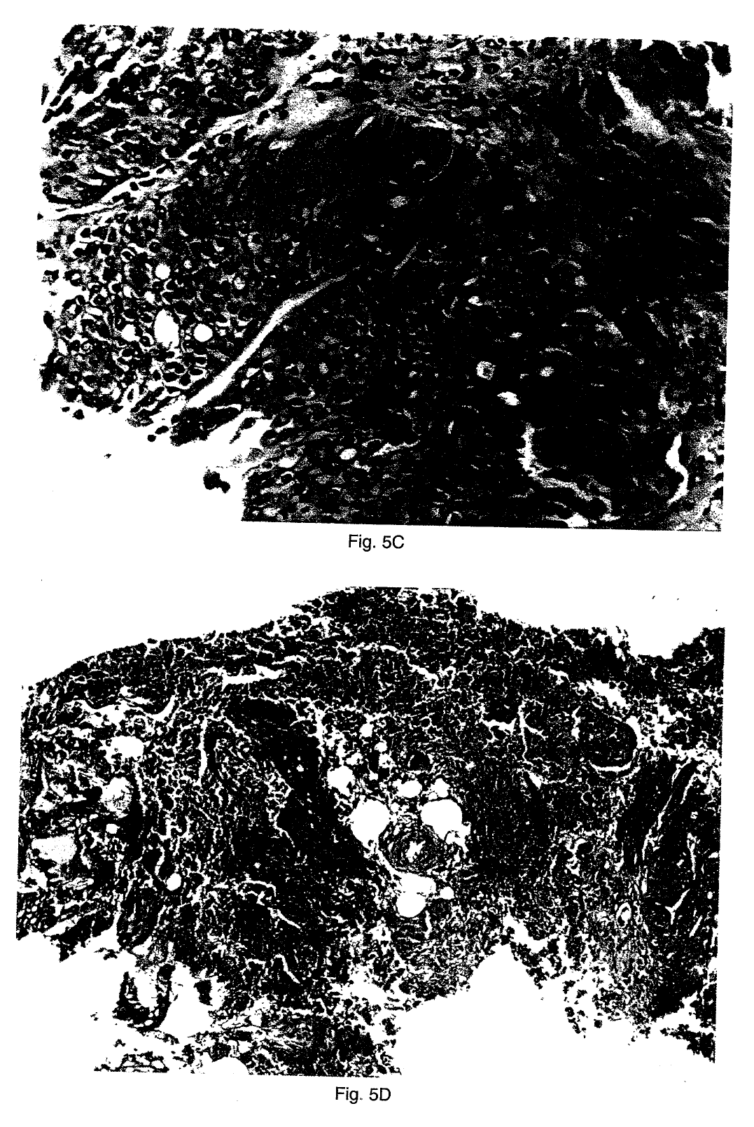

FIG. 5: in this figure the histological images of an endorectal prostatic biopsy (left lobule) of the DPC-1 orthotopic canine model at two months are assembled. FIG. 5a is a magnification multiplied by 4; it can be observed that the core is overrun almost totally by a carcinomatous tumorous proliferation. In FIG. 5b, the presence of a very undifferentiated carcinomatous proliferation formed in Ap is observed; the nucleoli are easily visible. It is a magnification multiplied by 25. FIG. 5c (magnification multiplied by 25) indicates the presence of a very undifferentiated carcinomatous tumorous proliferation formed in Ap; the presence of prostatic stroma (muscular fibres) is seen at the top of the image. FIG. 5d is an image with magnification multiplied by 10 of the biopsy in the same conditions as those of FIG. 5b.

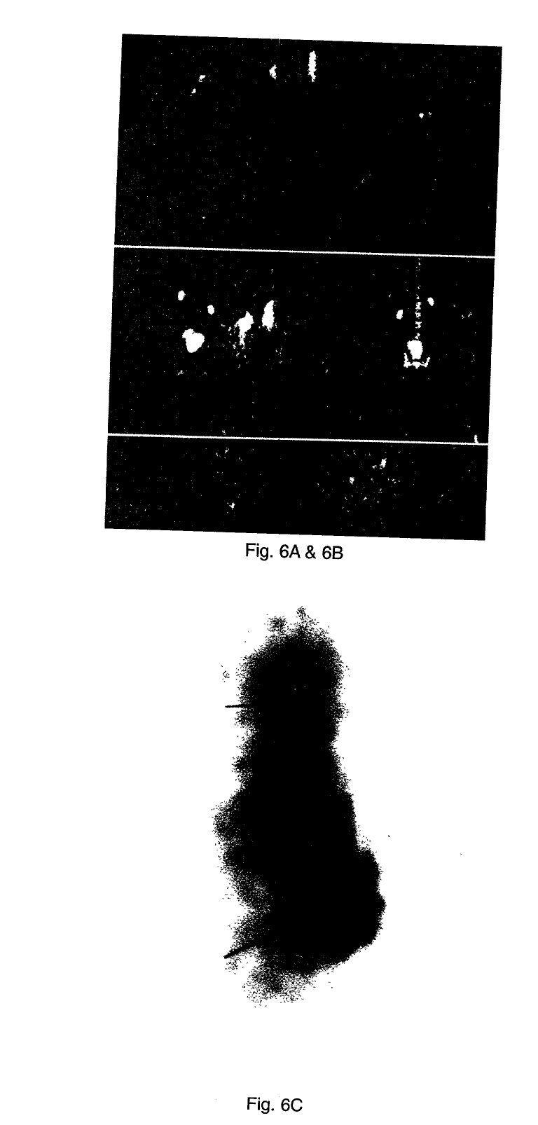

FIG. 6 shows an immuno-imaging image obtained with an anti-PSM-P12 antibody traced with iodine¹³¹I; the traced antibody is injected in a dog 12 weeks after an orthotopic injection of the DPC-1 line. As a control, the same dog was subjected to a bone isotope scanning with ⁹⁹Technetium. From left to right are shown:

a ventral view of the bone isotope scanning,

a dorsal view of the bone isotope scanning,

a ventral view in the immuno-imaging.

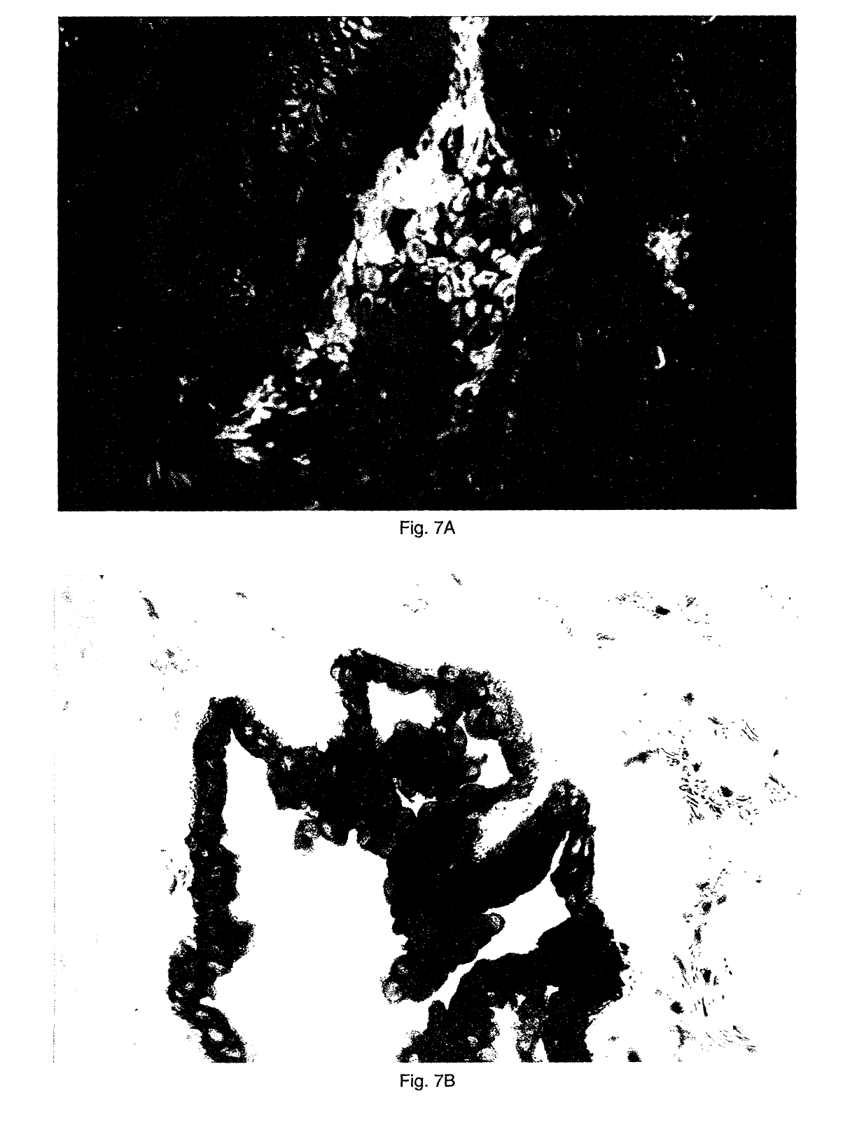

FIG. 7 shows the immunofluorescence tracing of a frozen section human normal and cancerous prostate, with the human anti-PSMA-P12 monoclonal antibody. The anti-human mouse monoclonal antibody dosed with 10 g/ml, is released by an anti-mouse rabbit polyclonal antibody traced with the fluorescence for the prostatic tumour (FIG. 7a) and the peroxidase for the normal prostate (FIG. 7b).

### EXAMPLE 1

Establishment of the DPC-1 Line

A non metastatic spontaneous prostatic tumour biopsy with at least 5 g is excised in an eleven year old Doberman Pincher dog then shredded into small pieces of about 3 mm³ and put into culture in 25 cm² flasks nia RPMI environment with 5% of calf foetal serum.

After twelve passages, the cells which have a monolayer growth are trypsinized and recovered.

Analysis by Immuno Histochemistry:

The experimental process of the immuno histochemistry analysis is described in O. Cussenot et al (1994), Experimental Cell Research, 214: 83-92.

Briefly, the cells are fixed with a methanol/acetone (2/1) mixture for 15 min. After three washes in PBS, in the presence of 0.1% BSA, the immunofluorescence is achieved by incubation at ambient temperature for 1 hour with the appropriate dilution of monoclonal or polyclonal antibodies. The cells are then incubated with the second antibody traced with the fluorescent. For each test, a negative control was carried out by using monoclonal or polyclonal antibodies having no chance of having an affinity for the cellular antigens but of the same sub-class of immunoglobulin.

Antibodies used

The antibodies used are shown in table 1 below.

| Parameters | Ac Type | Results |
|---|---|---|
| Cytokeratine 8 | M | Neg |
| Cytokeratine 14 | M | Pos |
| Cytokeratine 18 | M | Neg |
| Cytokeratine 19 | M | Pos |
| Vimentine | M | Pos |
| Ki67 | M | Pos |
| PSA | P | Pos |
| PAP | M | Neg |
| CGA | P | Neg |
| NSE | M | Neg |
| Androgen receptor | M | Neg |
| EGF receptor | M | Neg |
| FGF receptor | M | Neg |
| PSMA (PSM-P12) | M | Pos |

In the central column, the letter “M” indicates that it is a monoclonal antibody and the letter of a polyclonal antibody. In the third column, the term “Pos” indicates the existence of a positive reaction between the monoclonal or polyclonal antibody on the canine established line.

Immunofluorescence Results

The photographs of FIG. 1 are an illustration of the non ambiguous character of the reactivity of the DPC-1 line with the anti-cytokeratine 19 monoclonal (1a) or the human anti-PSA polyclonal (1b) antibody.

FIG. 2 clearly indicates that the new human anti PSMA-P12 monoclonal antibody recognises the DPC-1 canine line as well as the LNCaP human line (FIGS. 2a and 2bj respectively). The experiments indicated in example 5 below is illustrated by FIG. 6 showing that, by the same technology, this antibody reacts in the same way with a human prostate cancer.

Growth Characteristics

The doubling time of the population is measured in plates with 24 wells initially sowed with the density of 5×10³ cells per well. The number of cells in the 12 separate wells is determined 3 days successively by counting the nucleuses after a cellular lysis. Briefly, the cells are treated successively with a hypotonic buffer (10 mM Hepes, 1.5 mM Mgll₂), a lysis solution (3 ml of gacial acetic acid and 5 g MgCL₂ of diethylhexdecyl dimethylbromure of ammonia for 100 ml of distilled water) is fixed with 12.5% of formaldehyde in a PBS buffer. The counting of the nucleuses after cellular lysis appears to be the most reliable average for determining the number of cells, in so far as the cells of the DPC-1 line most often resist the trypcine-EDTA treatment.

The doubling time of the line is 27 hours and is not modified by the presence of di-hydrotesterone with different concentrations.

Cytogenetics:

The caryotype, evaluated by the characteristic methods (see references) indicates a hypoploidy, i.e. that the cells of the DPC-1 line contain 67 to 70 chromosomes (in place of 78).

The set of results above indicates that the DPC-1 line has all the essential characteristics of human prostatic tumour cells, i.e. a carotype of at least 60 chromosomes and a growth rate not modified by the addition of di-hydrotesterone, a positive recognition by the anti-PSA, PSMA-P12 anti-cytokeratine 19 monoclonal antibodies.

### EXAMPLE 2

Establishment of Stable Prostatic Tumour

2×10⁸ cells of the DPC-1 line are injected into a healthy dog prostate, in the left lobule, as appears in FIG. 3a, under control of a CT scanner. The cells are in suspension in 1 ml of RPMI without serum, and the dig used is an 9 year old golden retriever immunocompromised one week ago (J-7) with 3 mg/kg per day of cyclosporin administered orally.

The aspect and the formation of the tumour are followed by CT scanner image. After two weeks, the left lobe shows an affected aspect (FIG. 3b). After twelve weeks (photos 3c and 3d), and in biopsy (FIG. 4 and FIG. 5), after two months, there were obvious metastases, and the cyclosporin was stopped. The dog was killed after four months and an autopsy confirmed the lymphatic and even pulmonary metastases.

FIG. 5 shows the histological sections of the tumours after biopsy which confirm the existence of a tumour established in the left lobe of the prostate.

The immunofluorescence results obtained from the biopsy of this established canine cancer indicate the same reactivities with the monoclonal antibodies shown in table 1 above as the reactivities obtained with the DPC-1 line, and which show the characteristics of a human prostatic cancer.

### EXAMPLE 3

Tumorigenicity of the DPC-1 Line

Besides the grafting of the line in a canine prostate and the results described in example 2 above, the cells were injected in the feet pad of a nude mouse. The formation of a tumour in the lymphatic ganglions was observed in 100% of the cases after three to six weeks following the injection.

### EXAMPLE 4

Specificity of the PSMA-P12 Monoclonal Antibody

We recall that this monoclonal antibody was produced against a peptide of 20 amino acids localised in the extra cellular structure of the PSMA. It was selected for its capacities to trace the normal human prostatic epithelial cells by immuno histochemistry. FIGS. 2a and 2b indicate respectively the specificity of the anti PSMA P12, as much on the DPC-1 canine line as the LNCaP human line, which as such is a significant argument in favour of the validity of this line as a mouse tumorous prostatic line model. FIG. 6 shows that this antibody is specific as much on the human prostate cancers as on the canine prostate cancers, which also indicates that the animal model carrying the tumour established after grafting of the DPC-1 line cells is a model for which the tumour faithfully reflects the characteristics of a human tumour. It has indeed been observed that this antibody has the same specificity for the canine prostate (FIG. 6) as for the human prostate (FIG. 7).

All of the experiments described above indicate that the invention supplies the skilled worker with a direct means of achieving a prostatic tumour animal model useable in particular in pre-clinical research. These experiments also demonstrate that the new selected monoclonal antibody is a tool of choice in the diagnosis and the therapeutic follow up of cancer of the prostate.

1. An established cellular line obtained after separation and putting in culture of cells of a spontaneous prostatic tumour existing in an animal B, the cells of the aforesaid line being likely to be grafted in the prostate of an animal A of the same species or of a different species, and characterised:
in that it contains antigens recognised by the human anti-PSMA antibodies;
its carotype is at least 60 chromosomes.

2. The line in accordance with claim 1 recognised by the human anti-PSMA antibody is the PSM-P12 antibody registered with the CNCM the Aug. 6, 1999 under the n^{o} I-2280.

3. The line in accordance with one of the claims 1 or 2 carrying antigens recognised by the specific antibodies of the cytokerain 19 and the vimentin of human prostatic cells.

4. The line in accordance with any one of the claims 1 to 3 carrying antigens recognised by the antibodies directed against the human Ki67 antigen and/or against the human PSA antigen.

5. The line in accordance with any one of the claims 1 to 4 carrying antigens not recognised by the antibodies directed against the cytokerain 18 and/or against the androgenic receptors of prostatic epithelial cells.

6. The line in accordance with any one of the claims 1 to 5 for which the growth is not modified by the presence of variable levels of dihydrotesterone.

7. The line in accordance with the claims 1 to 6 characterised in that it is the DPC-1 line registered with the CNCM on the Aug. 6, 1999 under the n⁰ I-2279.

8. A production process of a non human mammalian animal A carrying a prostatic tumour comprising a grafting stage in the prostate of the aforesaid animal of 10⁷ to 10⁹ cells of an established cellular line in accordance with one of the claims 1 to 7.

9. The process in accordance with claim 8 wherein the animal A and the animal B are of the same species.

10. The process in accordance with claim 9 wherein the species is the dog.

11. The process in accordance with any one of the claims 8 to 10 characterised in that the animal is treated by an immunosuppressive drug simultaneous or sequential to the grafting of the cells.

12. The process in accordance with claim 11 wherein the immunosuppressive drug is the cyclosporin administered with a dose of between 1 and 10 mg per kg and per day to the animal at least two days before the aforesaid grafting.

13. A non human mammalian animal carrying a prostatic tumour, likely to be obtained by a process in accordance with one of the claims 8 to 12 by grafting in the prostate of the aforesaid animal of 10⁷ to 10⁹ cells of an established cellular line in accordance with one of the claims 1 to 7.

14. An animal in accordance with claim 13 characterised in that the prostatic tumour has the same characteristics as the cellular line in accordance with any one of the claims 1 to 7 and are characteristic of the prostate cancer in man.

15. A method for identifying a substance likely to treat a tumour of the prostate, the aforesaid method including the administration with effective doses of the aforesaid substance to an animal produced in accordance with any one of the claims 13 or 14 with the aforesaid substance, and the detection and the measurement, by comparison with a substance not suspected of having a therapeutic effect, of an effect on a reduction of the aforesaid tumour.

16. The method in accordance with claim 15 wherein the effect is detected and measured by immuno-imaging and/or by histological examination of a biopsy of the tumour.

17. The method in accordance with claim 15 wherein the detection and the measurement are carried out by coupling with a human anti PSMA antibody.

19. The method in accordance with claim 17 wherein the human anti PSMA monoclonal antibody is the PSM-P12 antibody registered with the CNCM on the Aug. 6, 1999 under the n^{o} I-2280 or a functional equivalent of that recognising the peptide 44-62 of the PSMA antigen.

19. The method in accordance with claim 15 wherein the substance is if the need arises coupled with a ligand of a specific receptor of tumorous cells of the prostate.

20. The method in accordance with one of the previous claims wherein an anti-idiotype antibody recognising the confirmational site of the coupling between the PSMA of specific antibodies of the PSMA is used to prevent the internalisation of these latter.

21. A process of obtaining a drug for the prophylaxis or the treatment of tumours of the prostate characterised in that, as an essential component of the aforesaid drug, a specific anti PSMA antibody from the N-terminal part of the extra cellular structure of the PSMA coupled with a substance identified in accordance with the method of claim 15 is made use of.

22. The process in accordance with claim 21 wherein the drug is a genic therapy drug and the substance chosen amongst the vectors or defective viruses used in this type of therapy.

23. The process in accordance with claim 21 wherein the substance is a chemical substance chosen in the group composed of GNRH hormones or one of their analogues.

24. The process in accordance with one of the claims 21 to 23 wherein the anti PSMA antibody is the PSM-P12 antibody registered with the CNCM on the Aug. 6, 1999 under the n^{o} I-2280 or a functional equivalent of that recognising the peptides 44-62 (cys-lys-ser-asn-glu-ala-thr-pro-lys-his-asn-met-lys-ala-phe-leu) of the PSMA antigen.

25. A PSM-P12 antibody registered with the CNCM on the Aug. 6, 1999 under the n^{o} I-2280 or a functional equivalent of that recognising the peptides 44-62 (cys-ser-asn-glu-ala-thr-pro-lys-met-lys-ala-phe-leu) of the PSMA antigen.

26. A coupling product between a specific monoclonal antibody and a substance of therapeutic or diagnostic interest of the cancer of the prostate.

27. The coupling product in accordance with claim 26, wherein the antibody is the PSM-P12 antibody or a functional equivalent of that recognising the peptides 44-62 (cys-lys-ser-asn-glu-ala-thr-pro-lys-his-asn-met-lys-ala-phe-leu) of the PSMA antigen.

28. The use of the PSM-P12 antibody registered with the CNCM on the Aug. 6, 1999 under the n^{o} I-2280 or a functional equivalent of that recognising the peptides 44-62 (cys-lys-ser-asn-glu-ala-thr-pro-lys-his-asn-met-lys-ala-phe-leu) of the PSMA antigen in a targetting process on tumorous cells of the prostate of substances of diagnostic or therapeutic interest.
